(19) United States
(12) Patent Application Publication
(10) Pub. No.: US 2008/0145318 A1
(43) Pub. Date: Jun. 19, 2008

(54) ATOMOXETINE FORMULATIONS AND ASSOCIATED METHODS

(76) Inventor: Kamal K. Midha, London (GB)

Correspondence Address: Hedman and Costigan PC, 1185 Avenue of the Americas, Ste 2003, New York, NY, 10036, US

(21) Appl. No.: 11/610,330

(22) Filed: Dec. 13, 2006

(51) Int. Cl. A61K 31/135; A61K 31/439; A61K 9/70; A61K 9/12

(52) U.S. Cl. 424/45; 514/651; 514/305; 424/449

(57) ABSTRACT

Methods and formulations for delivering atomoxetine compounds that minimize drug metabolism and thus increase the effectiveness of the drug are disclosed. The in vivo potency of the atomoxetine compound may be maximized by minimizing the in vivo conversion of the atomoxetine compound to an atomoxetine compound metabolite.

### FIELD OF THE INVENTION

The present invention relates to atomoxetine prodrug and metabolite formulations and methods for the treatment of various medical conditions in a subject. Accordingly, this invention involves the fields of chemistry, pharmaceutical sciences, medicine and other health sciences.

### BACKGROUND OF THE INVENTION

Physiological variability between individuals can often complicate the administration of various pharmaceuticals. For example, metabolic conversion variability between individuals can be problematic to the administration of pharmaceuticals, both in terms of variability in the amount of active drug available to exert a therapeutic effect, and variability of experienced side effects. These variable pharmacological effects between individuals can create dosing challenges, particularly for those drugs that affect behavior or those that require fairly specific blood serum level ranges for proper therapeutic effectiveness.

Depending on the drug being delivered, many side effects may be at least partially caused by metabolized drug products. Side effects are thus generally exacerbated in individuals that require higher doses of a drug due to increased drug metabolism. In addition to increasing serum levels of the active drug, increasing the administered dose tends to increase the concentration of drug metabolites in the blood due to increased drug metabolism. As such, the severity of such adverse effects also generally increases if the metabolites are responsible for the adverse effects.

As stated previously, metabolic variability between individuals can also be problematic to the administration of many drugs that need to achieve a precise range of blood serum levels in order to attain or optimize the intended therapeutic effect. In these cases, blood serum levels between individuals that metabolize the drug at different rates can vary dramatically. Those that metabolize quickly will experience a rapid decline of the drug in blood serum levels, while those that metabolize more slowly retain higher levels of the drug for much longer periods. As such, it can be difficult to prescribe and monitor the therapeutic actions of a drug across individuals.

Considerable variability may also exist between individuals with regards to side effects experienced from a particular drug. Pharmaceutical compounds are often metabolized into multiple metabolites upon administration. As such, in addition to variation among individuals due to tolerance of a metabolite, i.e. variability in the plasma level of the metabolite to cause side effect symptoms, there also may be variability among individuals as to which metabolites cause side effects. This becomes particularly problematic when the administered dose is increased to overcome metabolic variability. In these situations, plasma levels of all metabolites may increase, leading to further increases in experienced side effects.

Atomoxetine is an example of a drug that may exhibit such metabolic problems associated with its administration. Atomoxetine is a selective norepinephrine reuptake inhibitor (SNRI) that is often used in the treatment of attention-deficit/hyperactivity disorder (ADHD), and is commercially available as the oral formulation Strattera® from Eli Lilly Co. The precise mechanism by which atomoxetine exerts its effects in ADHD is unknown, however ex vivo uptake and neurotransmitter depletion studies suggest that it may be related to selective inhibition of the pre-synaptic norepinephrine transporter.

Atomoxetine is metabolized primarily by oxidative metabolism through the cytochrome P450 2D6 (CYP2D6) enzymatic pathway and subsequently eliminated through glucuronidation. At least two phenotypes of drug metabolism associated with CYP2D6 have been identified in the population, one exhibiting normal activity and one exhibiting reduced activity. In individuals having normal activity in the CYP2D6 pathway, atomoxetine has a plasma half-life of about 5 hours. In individuals that are part of the population segment having reduced activity in the CYP2D6 pathway, and thus are poor metabolizers of the drug, atomoxetine has a half-life of about 24 hours. As such, the administration of atomoxetine can be difficult without prior testing of individuals to determine the rate at which they metabolize through the CYP2D6 enzymatic pathway.

In view of the foregoing, compositions and methods for administering atomoxetine that reduce problems associated with drug metabolism and side effects continue to be sought.

### SUMMARY OF THE INVENTION

Accordingly, the present invention provides atomoxetine agent formulations and their methods of use which alleviate the foregoing issues. A variety of atomoxetine agents are encompassed by various aspects of the present invention, including atomoxetine metabolites and atomoxetine prodrugs. In some aspects, the atomoxetine agent may be any atomoxetine prodrug or metabolite that has been modified to alter the rate at which the drug is metabolized. Such a modification may be accomplished, inter alia, by blocking the phenoxy 4 position of the atomoxetine agent, for example.

In one aspect, the present invention provides a method for treating or preventing a condition in a subject for which atomoxetine is effective by administering a therapeutically effective amount of an atomoxetine agent, rather than atomoxetine itself, to the subject. Numerous atomoxetine agents are considered be within the present claim scope, including various known atomoxetine prodrugs or metabolites. Additionally, in one aspect the atomoxetine agent may be a compound according to Formula I:

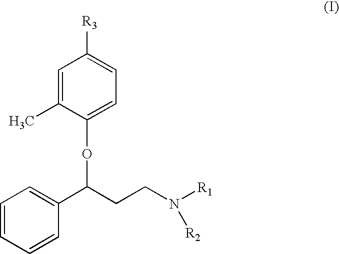

or an isomer, stereoisomer, enantiomer, tautomer, analog, salt, or combination thereof, where R₁ and R₂ are independently a branched or unbranched C₁LC₃ alkyl or a branched or unbranched C₁-C₃ N-oxide, including respective tertiary oxides, R₃ is —H or —OR₄, and R₄ can be alkyl or branch chain alkyl of C1-C18, a substituted or unsubstituted phenyl ring, —C₆H₉O₆, or —H. Depending on the particular atomoxetine agent involved, R₁ and R₂ may be the same or R₁ and R₂ may be different.

In another aspect, the atomoxetine agent may be a compound according to Formula II:

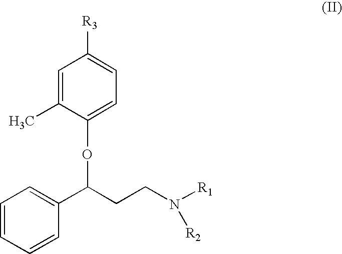

or an isomer, stereoisomer, enantiomer, tautomer, analog, salt, or combination thereof, where R₁ is —H, R₂ is —CH₃ or —H, R₃ is —H or —OR₄ when R₂ is —H and R₃ is —OR₄ when R₂ is —CH₃, and R₄ can be alkyl or branch chain alkyl of C1-C18, a substituted or unsubstituted phenyl ring, —C₆H₉O₆, or —H. As with Formula I, R₁ and R₂ may be the same or R₁ and R₂ may be different.

Various conditions may be treated or prevented by the methods and formulations of the present invention. Such conditions may include, without limitation, attention-deficit/hyperactivity disorders (ADHD), asthma, allergic rhinitis, cognitive failure, tic disorders, depression, resistant depression with psychotic features, motor deficit after stroke, memory disorders, obesity, Tourette's syndrome, traumatic brain injury, bipolar disorder, anxiety, narcolepsy, nocturnal enuresis, fibromyalgia syndrome, schizophrenia, post traumatic stress disorder, and combinations and related disorders thereof.

Atomoxetine agent formulations for treating or preventing a condition are also provided. In one aspect such a formulation may include a therapeutically affective amount of an atomoxetine agent in combination with a pharmaceutically acceptable carrier. The dosage form of the formulation may include oral, transdermal, parenteral, or any other known delivery means.

In various aspects, the atomoxetine agent formulations of the present invention may be provided as an oral dosage form. Any pharmaceutically acceptable oral formulation and method for administering an atomoxetine agent that does not interfere with the drug's therapeutic effects may be used for achieving the desired aspects of the present invention. The oral dosage forms of the present invention may take a variety of well-known delivery formulations, including but not limited to, tablets, capsules, caplets, powders, pellets, granules, syrups, elixirs, etc.

The transdermal formulations of the present invention may take numerous specific embodiments. In one aspect, the formulation may be a transdermal patch. Transdermal patches may include any type of patch known to one skilled in the art, including transdermal matrix patches, liquid reservoir patches, etc. Further examples include transmucosal formulations, such as buccal and sublingual tablets or adhesive films. In another aspect, the transdermal formulation may be a topical formulation. Topical formulations may include, without limitation, creams, lotions, ointments, gels, pastes, mousses, aerosols, sprays, waxes, balms, suppositories, and mixtures or combinations thereof. Any one of a number of specific ingredients may be used in order to provide a specifically desired transdermal formulation, such as diluents, excipients, emollients, plasticizers, skin irritation reducing agents, stabilizing compounds, and mixtures thereof.

The potency of an atomoxetine agent may be enhanced by administering a P450-mediated reaction inhibitor to the subject. Though various P450-mediated reaction inhibitors may prove to be useful in increasing potency when administered in association with an atomoxetine agent, an inhibitor of the CYP2D6 enzymatic pathway may be particularly effective. A P450-mediated reaction inhibitor can be administered either prior to, concurrently with, or following the atomoxetine agent. Such an inhibitor may also be administered both prior to and following the atomoxetine agent. Also, in one aspect the P450-mediated reaction inhibitor and the atomoxetine agent may be administered as a single composition.

In a specific embodiment of the present invention, a transdermal atomoxetine agent formulation is provided having a pressure sensitive acrylic polymer in an amount of about 60 to about 90% w/w of the transdermal formulation, N-ethylatomoxetine in an amount of about 0.1 to about 50% w/w of the transdermal formulation, polyvinylpyrrolidone in an amount of about 10% w/w of the transdermal formulation, a penetration enhancer in an amount of about 5 to about 20% w/w of the transdermal formulation, and quinidine in an amount of about 0.1% w/w or greater of the transdermal formulation.

In another specific embodiment, an oral atomoxetine agent formulation is provided having polyethylene glycol in an amount of about 20 to about 25% w/w of the oral formulation, N-ethylatomoxetine in an amount of about 0.1 to about 40% w/w of the oral formulation, and quinidine in an amount of about 0.1% w/w or greater of the oral formulation.

### DETAILED DESCRIPTION

### Definitions

In describing and claiming the present invention, the following terminology will be used in accordance with the definitions set forth below.

The singular forms “a,” “an,” and, “the” include plural referents unless the context clearly dictates otherwise. Thus, for example, reference to “an adhesive” includes reference to one or more of such adhesives, and reference to “an excipient” includes reference to one or more of such excipients.

As used herein, the term “atomoxetine agent” refers to any compound that is functionally similar to atomoxetine, but excluding atomoxetine. Such compounds may include, without limitation, those recited above, as well as other metabolites, derivatives, salts, prodrugs, analogs, isomers, etc.

As used herein, the terms “atomoxetine” and “tomoxetine” may be used interchangeably, both of which refer to a compound having the general chemical structure:

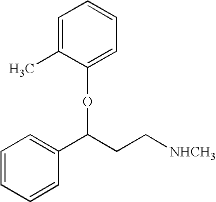

Atomoxetine is well known in the art, and is also known chemically as (−)-N-methyl-3-phenyl-3-(o-tolyloxy)-propylamine. This selective norepinephrine reuptake inhibitor is commercially available as atomoxetine HCl under the brand name Strattera® from Eli Lilly Co. Numerous metabolites of atomoxetine are known having varying physiological activities. For example, atomoxetine is converted in vivo into the active metabolite 4-hydroxyatomoxetine (4HA), primarily by aromatic hydroxylation via the cytochrome P450 2D6 (CYP2D6) enzymatic pathway. Atomoxetine is also converted in vivo into the active metabolite N-desmethylatomoxetine (NDA), primarily through the cytochrome P450 2C19 (CYP2C19) enzymatic pathway.

As used herein in, “4-hydroxyatomoxetine” and “4HA” may be used interchangeably, and refer to a compound having the general chemical structure:

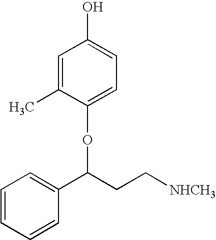

4HA possesses similar inhibitory activity to the norepinephrine reuptake transporter as atomoxetine, and is also a pharmacologically active serotonin reuptake inhibitor. This metabolite appears to show little affinity to other receptor systems. 4HA is metabolized through glucuronidation to form the inactive metabolite 4-hydroxyatomoxetine-O-glucuronide (4HAO-G), which is further metabolized and/or eliminated from the body. 4HAO-G is formed to a large extent presystemically through first pass hepatic metabolism mechanisms in the gut and liver when atomoxetine agents are administered orally. Additionally, isomers of 4HA are also included within this definition. For example, one negative isomer that may be included is defined as N-methyl-3-phenyl-3-2-methyl-4-hydroxyphenyl propylamine.

As used herein in, “N-desmethylatomoxetine” and “NDA” may be used interchangeably, and refer to a compound having the general chemical structure:

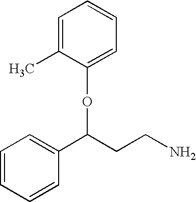

NDA is less active at inhibiting the norepinephrine reuptake transporter compared to atomoxetine. This metabolite appears to show little affinity to other receptor systems. With regard to metabolism, NDA is hydroxylated at the 4 position of the phenoxy ring, glucuronidated, and subsequently eliminated from the body.

As used herein in, “N-desmethyl-4-hydroxyatomoxetine” and “4H-NDA” may be used interchangeably, and refer to a compound having the general chemical structure:

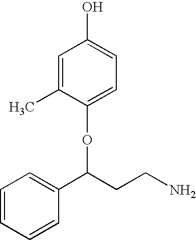

4H-NDA is less active at inhibiting the norepinephrine reupiake transporter compared to atomoxetine. This metabolite also appears to show little affinity to other receptor systems. With regard to metabolism, 4H-NDA is glucuronidated and subsequently eliminated from the body.

As used herein, “N-methylatomoxetine” refers to a compound having the general chemical structure:

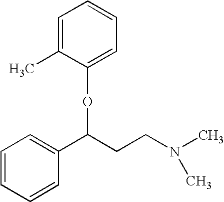

N-methylatomoxetine is a prodrug that is converted in vivo into atomoxetine.

As used herein, “N-methylatomoxetine N-oxide” refers to a compound having the general chemical structure:

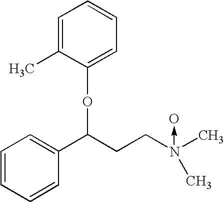

N-methylatomoxetine N-oxide is a prodrug that is converted in vivo into atomoxetine.

As used herein, “N-ethylatomoxetine” refers to a compound having the general chemical structure:

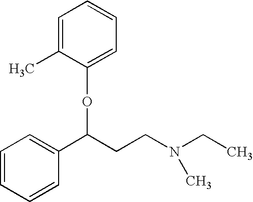

N-ethylatomoxetine is a prodrug that is converted in vivo into atomoxetine.

The above described atomoxetine agents are meant to be exemplary, and as such are not meant to be a limiting of acceptable atomoxetine agents. Thus the scope of the present invention is not limited by these structures, but would include those related compounds that would be apparent to one of ordinary skill in the art once in possession of this disclosure.

As used herein, the “phenoxy 4 position” refers to the 4^{th} carbon of the phenoxy group of an atomoxetine agent. As an illustration, the phenoxy 4 position is marked by an X in the following exemplary structure:

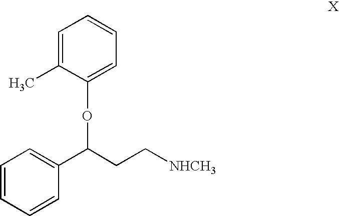

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts.

The term “metabolite” refers to a form of a compound obtained in a human or animal body by action of the body on the administered form of the compound. One non-limiting example may include a de-methylated analog of a compound bearing a methyl group which is obtained in the body after administration of the methylated compound. Such de-methylation is a result of action by the body on the methylated compound. Additionally, metabolites may themselves have biological activity.

As used herein, an “atomoxetine metabolite” refers to any metabolite that may be formed by metabolism of atomoxetine or an atomoxetine agent. Atomoxetine metabolites may include, without limitation, 4-hydroxyatomoxetine, 4-hydroxyatomoxetine-O-glucuronide, N-desmethylatomoxetine, N-desmethyl-4-hydroxyatomoxetine, isomers, stereoisomers, enantiomers, tautomers, analogs, metabolites, salts, or combinations thereof. Various active and inactive metabolites or prodrugs of atomoxetine compounds are known, and it is intended that the administration of all such active metabolites be included in the scope of the present invention, as well as the administration of inactive metabolites that may be metabolized into an active form.

As used herein “prodrug” refers to a molecule that will convert into an active parent drug and/or active metabolites of the present invention in vivo when the prodrug is administered to a subject. Prodrugs themselves can also be pharmacologically active, and therefore are also expressly included within the definition of an “active agent,” as described above. Because prodrugs are known to enhance numerous desirable qualities of pharmaceuticals (i.e., solubility, bioavailability, manufacturing, etc.) the compounds of the present invention may be delivered in prodrug form. Prodrugs of the present invention may be prepared by any means known to one of ordinary skill in the art, including, but not limited to, modifying functional groups present in the compound in such a way that the modifications are cleaved, either in routine manipulation or in vivo, to the parent compound. The transformation in vivo may be, for example, the result of a metabolic process, such as chemical or enzymatic hydrolysis, or reduction or oxidation of a susceptible functionality.

As used herein, an “atomoxetine prodrug” refers to any prodrug that may be metabolized to atomoxetine or to an atomoxetine metabolite. Atomoxetine prodrugs may include, without limitation, N-methylatomoxetine, N-ethylatomoxetine, N-propylatomoxetine, N,N-diethyl-3-phenyl-3-(o-tolyloxy)-propylamine, N,N-dipropyl-3-phenyl-3-(o-tolyloxy)-propylamine, N-ethyl-N-propyl-3-phenyl-3-(o-tolyloxy)-propylamine, N-methyl-N-isopropyl-propylamine, N-methyl-N-isobutyl-propylamine, N-methyl-N-tertbutyl-propylamine, and their N-oxides, stereoisomers, enantiomers, tautomers, analogs, prodrugs, salts, or combinations thereof. Various active and inactive prodrugs of atomoxetine compounds are known, and it is intended that the administration of such prodrugs be included in the scope of the present invention.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals.

As used herein, “blood level” may be used interchangeably with terms such as blood plasma concentration, plasma level, plasma concentration, serum level, serum concentration, serum blood level and serum blood concentration.

“Administration,” and “administering” refer to the manner in which an active agent is presented to a subject. Administration can be accomplished by various art-known routes such as oral, parenteral, transdermal, inhalation, implantation, etc.

The term “oral administration” represents any method of administration in which an active agent can be administered by swallowing, chewing, or sucking an oral dosage form. Such solid or liquid oral dosage forms are traditionally intended to substantially release and or deliver the active agent in the gastrointestinal tract beyond the mouth and/or buccal cavity. Examples of solid dosage forms include conventional tablets, capsules, caplets, etc., which do not substantially release the drug in the mouth or in the oral cavity.

As used herein, “oral dosage form” refers to a formulation that is ready for administration to a subject through the oral route of administration. Examples of known oral dosage forms, include without limitation, tablets, capsules, caplets, powders, pellets, granules, etc. Such formulations also include multilayered tablets wherein a given layer may represent a different drug. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Tablets and caplets may be scored to facilitate division of dosing. Alternatively, the dosage forms of the present invention may be unit dosage forms wherein the dosage form is intended to deliver one therapeutic dose per administration.

The term “non-oral administration” represents any method of administration in which an active agent is not provided in a solid or liquid oral dosage form. It is appreciated that many oral liquid dosage forms such as solutions, suspensions, emulsions, etc., and some oral solid dosage forms may release some of the drug in the mouth or in the oral cavity during the swallowing of these formulations. However, due to their very short transit time through the mouth and the oral cavities, the release of drug from these formulations in the mouth or the oral cavity is considered de minimus or insubstantial. Thus, buccal patches, adhesive films, sublingual tablets, and lozenges that are designed to release the drug in the mouth are non-oral compositions for the present purposes.

In addition, it is understood that the term “non-oral” includes parenteral, transdermal, inhalation, implant, and vaginal or rectal formulations and administrations. Further, implant formulations are to be included in the term “non-oral,” regardless of the physical location of implantation. Particularly, implantation formulations are known which are specifically designed for implantation and retention in the gastrointestinal tract. Such implants are also considered to be non-oral delivery formulations, and therefore are encompassed by the term “non-oral.”

“Parenteral administration” can be achieved by injecting a drug composition intravenously, intra-arterially, intramuscularly, intrathecally, subcutaneously, etc.

As used herein, “transdermal” refers to the route of administration taken by a drug that is applied to and absorbed through an area of skin. In some aspects, the skin may be substantially unbroken. Thus the terms “transdermal formulation” and “transdermal composition” can be used interchangeably, and refer to formulations or compositions that are applied to a surface of the skin and transdermally absorbed. Examples of transdermal formulations include but are not limited to, ointments, creams, gels, transdermal patches, sprays, lotions, mousses, aerosols, nasal sprays, buccal and sublingual tablets and tapes or adhesives, vaginal rings, and pastes. The term “transdermal administration” thus refers to the transdermal application of a formulation or composition. Transdermal administration can be accomplished by applying, pasting, rolling, attaching, pouring, pressing, rubbing, etc., of a transdermal preparation or formulation onto a skin or mucosal surface of a subject. These and additional methods of administration are well-known in the art.

The terms “transdermal delivery system,” “transdermal patches” or simply “patches” refer to a polymeric matrix or liquid reservoir type of transdermal delivery device which is used to transdermally deliver defined doses of a substance, over a specific application period.

By the term “matrix”, “matrix system”, or “matrix patch” is meant a composition comprising an effective amount of a drug dissolved or dispersed in a polymeric phase, often a pressure sensitive adhesive, which may also contain other ingredients, such as a permeation enhancers, skin irritation reducing agents, excipients, plasticizers, emollients, and other optional ingredients. This definition is meant to include embodiments wherein such polymeric phase is laminated to a pressure sensitive adhesive or used within an overlay adhesive.

The general structure of a matrix-type patch is known to those skilled in the art. Such structure typically includes a drug-impermeable occlusive backing laminated to the distal side of a solid or semisolid matrix layer comprised of a homogeneous blend of the drug, a polymeric pressure sensitive adhesive carrier, and optionally one or more skin permeation enhancers, and a temporary peelable release liner adhered to the proximal side of the matrix layer. In use, the release liner is removed prior to application of the patch to the skin. Matrix patches are known in the art of transdermal drug delivery. Examples without limitation, of adhesive matrix transdermal patches are those described or referred to in U.S. Pat. Nos. 5,985,317, 5,783,208, 5,626,866, 5,227,169, 5,122,383 and 5,460,820 which are incorporated by reference in their entirety.

Additionally, the general structure of a liquid reservoir system (LRS) type patch is also known. Such patches typically comprise a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. The drug and any penetration enhancers are contained in the fluid in desired amounts. For application, a peelable release liner is removed and the patch is attached to the skin surface. LRS patches are known in the art of transdermal drug delivery. Examples without limitation, of LRS transdermal patches are those described or referred to in U.S. Pat. Nos. 4,849,224, 4,983,395, which are incorporated by reference in their entirety.

“Topical formulation” means a composition in which an active agent may be placed for direct application to a skin surface and from which an effective amount of the active agent is released.

The terms “skin,” “skin surface,” “derma,” “epidermis,” and similar terms are used interchangeably herein, and refer to not only the outer skin of a subject comprising the epidermis, but also to mucosal surfaces to which a drug composition may be administered. Examples of mucosal surfaces include the mucosal of the respiratory (including nasal and pulmonary), oral (mouth and buccal), vaginal, introital, labial, and rectal surfaces. Hence the terms “transdermal” encompasses “transmucosal” as well.

As used herein, “enhancement,” “penetration enhancement,” or “permeation enhancement,” refer to an increase in the permeability of the skin to a drug, so as to increase the rate at which the drug permeates through the skin. Thus, “permeation enhancer,” “penetration enhancer,” or simply “enhancer” refers to an agent, or mixture of agents that achieves such permeation enhancement. Several compounds have been investigated for use as penetration enhancers. See, for example, U.S. Pat. Nos. 5,601,839; 5,006,342; 4,973,468; 4,820,720; 4,006,218; 3,551,154; and 3,472,931. An index of permeation enhancers is disclosed by David W. Osborne and Jill J. Henke, in their publication entitled Skin Penetration Enhancers Cited in the Technical Literature, published in “Pharmaceutical Technology” (June 1998), which is incorporated by reference herein.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference. Thus, an “effective amount” of an enhancer refers to an amount sufficient to increase the penetration of a drug through the skin to a selected degree. Methods for assaying the characteristics of permeation enhancers are well-known in the art. See, for example, Merritt et al., “Diffusion Apparatus for Skin Penetration,” J. of Controlled Release 61 (1984), incorporated herein by reference in its entirety.

As used herein, “pharmaceutically acceptable carrier” and “carrier” may be used interchangeably, and refer to any inert and pharmaceutically acceptable material that has substantially no biological activity, and makes up a substantial part of the formulation. The carrier may be polymeric, such as an adhesive, or non-polymeric, and is generally admixed with other components of the composition (e.g., drug, binders, fillers, penetration enhancers, anti-irritants, emollients, lubricants, etc., as needed) to comprise the formulation.

The term “admixed” means that the drug and/or other ingredients can be dissolved, dispersed, or suspended in the carrier. In some cases, the drug may be uniformly admixed in the carrier.

As used herein, “substantially” when used in reference to a quantity or amount of a material, or a specific characteristic thereof, refers to an amount that is sufficient to provide an effect that the material or characteristic was intended to provide. The exact degree of deviation allowable may in some cases depend on the specific context. Similarly, “substantially free of” or the like refers to the lack of an identified element or agent in a composition. Particularly, elements that are identified as being “substantially free of” are either completely absent from the composition, or are included only in amounts which are small enough so as to have no measurable effect on the composition.

The terms “adverse drug experience” and “side effects” may be used interchangeably, and refer to any adverse event associated with the use of a drug in a subject, including the following: an adverse event occurring in the course of the use of a drug product in professional practice; an adverse event occurring from drug overdose whether accidental or intentional; an adverse event occurring from drug abuse; an adverse event occurring from drug withdrawal; and any failure of expected pharmacological action. The adverse drug experience may lead to a substantial disruption of a person's ability to conduct normal life functions. In some instances, the adverse drug experience may be serious or life threatening. Additionally, minor unintended physiological effects associated with the administration of a drug would also be considered to be within the scope of these terms.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 11 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc.

This same principle applies to ranges reciting only one numerical value. Furthermore, such an interpretation should apply regardless of the breadth of the range or the characteristics being described.

### THE INVENTION

The present invention involves the use of various metabolites of atomoxetine and atomoxetine prodrugs for administration to a subject in order to achieve therapeutic effects similar to the administration of atomoxetine. In some cases, the administration of a metabolite or a prodrug may provide improved dosing variability over that of atomoxetine.

As has been described herein, metabolic conversion variabilities between individuals may affect pharmacokinetic profiles, and thus may affect therapeutic activity for atomoxetine agents. Variabilities can arise from various factors, such as CYP2D6 genetic diversity in a population or from drug-drug interactions with potent CYP2D6 inhibitors. Such metabolic variabilities may be minimized through the administration of atomoxetine prodrugs and/or metabolites which may bypass the CYP2D6 enzymatic pathway. For example, CYP2D6 does not appear to be a major metabolizer of 4HA, and as such, the administration of such a metabolite may bypass the CYP2D6 enzymatic pathway altogether. Additionally, the transdermal administration of atomoxetine prodrugs and/or metabolites may also bypass first pass hepatic metabolism, and thus minimize metabolic variability between individuals. As such, the in vivo potency and variability of an atomoxetine agent may be maximized by minimizing drug metabolism. The oral and non-oral administration of an atomoxetine prodrug or metabolite may also reduce the drug's overall metabolic burden due to a comparatively lower administered dosage. The selection of a particular prodrug or metabolite of atomoxetine may also allow for decreased dosage variability for those subjects that are difficult to provide proper dosages to as a result of metabolic or side effect issues.

The present invention can be used to deliver a wide variety of atomoxetine agents to a subject. The inventors have found that the administration of certain prodrugs and metabolites of atomoxetine may be particularly effective in treating ADHD and other disorders for which atomoxetine has found uses, in some cases due to their avoidance of certain primary hepatic metabolic mechanisms. Any prodrug known to one of ordinary skill in the art that may be metabolized into atomoxetine upon administration to a subject is considered to be within the scope of the present invention. Specific examples may include, without limitation, N-methylatomoxetine, N-ethylatomoxetine, N-propylatomoxetine, N,N-diethyl-3-phenyl-3-(o-tolyloxy)-propylamine, N,N-dipropyl-3-phenyl-3-(o-tolyloxy)-propylamine, N-ethyl-N-propyl-3-phenyl-3-(o-tolyloxy)-propylamine, N-methyl-N-isopropyl-propylamine, N-methyl-N-isobutyl-propylamine, N-methyl-N-tertbutyl-propylamine, and their N-oxides, stereoisomers, enantiomers, tautomers, analogs, metabolites, prodrugs, salts, or combinations thereof. In one specific aspect, the atomoxetine agent may be N-ethylatomoxetine. In another specific aspect, the atomoxetine agent may be N,N-diethyl-3-phenyl-3-(o-tolyloxy)-propylamine.

Various atomoxetine metabolizes may also be therapeutically effective in a subject. As such, any atomoxetine metabolite that exhibits a desired therapeutic effect on a subject would be considered to be within the scope of the present invention. Examples of specific atomoxetine metabolites include, without limitation, atomoxetine, 4-hydroxyatomoxetine (4HA), N-desmethylatomoxetine (NDA), 4-hydroxyatomoxetine-O-glucuronide, N-desmethyl-4-hydroxyatomoxetine, N-desmethyl-4-hydroxyatomoxetine-O-glucuronide, isomers, stereoisomers, enantiomers, tautomers, analogs, prodrugs excluding atomoxetine, salts, or combinations thereof. In one specific aspect, the atomoxetine metabolite may be 4HA. In another specific aspect, the atomoxetine metabolite may be NDA. Due its lower inhibitory activity, NDA may be particularly useful for those situations where lower inhibition of the norepinephrine transporter may be desired. In yet another specific aspect, the atomoxetine metabolite may be 4-hydroxyatomoxetine-O-glucuronide.

In one aspect of the present invention, the atomoxetine agent may be an atomoxetine agent blocked at the phenoxy 4 position. Because atomoxetine agents, particularly 4HA, are glucuronidated via the phenoxy 4 position and eliminated from the body, blocking this position may increase the potency of the atomoxetine agent by reducing drug metabolism and subsequent elimination. Any mechanism of blocking the phenoxy 4 position known to one skilled in the art is considered to be within the scope of the present invention. For example, the phenoxy 4 position may be blocked with an ester moiety. Such moieties may include, without limitation, methoxy, ethoxy, etc., such as branched alkyl groups with of C₃-C₈, or OC₄OH₉.

The atomoxetine agent formulations of the present invention may be provided as an oral dosage form. Any pharmaceutically acceptable oral formulation and method for administering an atomoxetine agent that does not interfere with the drug's therapeutic effects may be used for achieving the desired aspects of the present invention.

In one aspect, the atomoxetine formulation may be a solid oral dosage form of an atomoxetine agent. Such an administration form will generally include a therapeutically effective amount of the atomoxetine agent in a substantially solid pharmaceutically acceptable carrier. The solid dosage form, upon oral administration, provides a therapeutically effective blood serum level of atomoxetine and/or an atomoxetine metabolite to a subject. The atomoxetine agent dosage forms of this invention may be prepared by injection molding techniques, or any other manufacturing method known to one skilled in the art.

The solid oral dosage forms of the present invention can be processed into dosage forms such as tablets, capsules, caplets, powders, encapsulated pellets, encapsulated granules, encapsulated powders, etc. These dosage forms can be coated with a polymeric or other art-known coating material to achieve, for example, greater stability on the shelf or in the gastrointestinal tract, or to achieve control over drug release. Such coating techniques and materials used therein are well-known in the art. For example, cellulose acetate phthalate, polyvinyl acetate phthalate, hydroxypropylmethyl cellulose phthalate, methacrylic acid-methacrylic acid ester copolymers, cellulose acetate trimellitate, carboxymethylethyl cellulose, and hydroxypropylmethyl cellulose acetate succinate, among others, can be used to achieve enteric coating. Mixtures of waxes, shellac, zein, ethyl cellulose, acrylic resins, cellulose acetate, silicone elastomers, etc., can be used to achieve sustained release coating. See, for example, Remington: The Science and Practice of Pharmacy 20^{th} ed. (2000), Chapter 46, which is incorporated by reference, for other types of coatings, techniques, and equipment.

Various pharmaceutically acceptable carriers are known to one of ordinary skill in the art and are considered to be within the scope of the present invention. Examples include, without limitation, polyethylene glycols, polyvinylpyrrolidone, a cellulose ether, carboxyalkyl celluloses (carboxymethyl cellulose, carboxyethyl cellulose, etc), etc., or mixtures thereof.

In one aspect, a solid oral dosage formulation may include a substantially solid polyethylene glycol carrier in combination with the atomoxetine agent. Any amount of carrier that is required in order to achieve a formulation with specifically desired characteristics may be used. However, in one aspect, the substantially solid polyethylene glycol carrier may be from about 30% w/w to about 80% w/w of the oral dosage formulation. In an additional aspect, the substantially solid polyethylene glycol carrier may be from about 50% w/w to about 80% w/w of the oral dosage formulation. In another aspect of the present invention, the substantially solid polyethylene glycol carrier may be from about 60% w/w to about 80% w/w of the oral dosage form. In yet another aspect, the substantially solid polyethylene glycol carrier may be about 70% w/w of the oral dosage form.

Polyethylene glycol is available in various grades under several trademarks including CARBOWAX® PEG 200, 300, 400, 540 BLEND, 900, 1000, 1450, 3350, 4000, 4600, 8000 and compound 20M from Union Carbide Co., USA and POLYGLYCOLS E® series from Dow chemical Co., USA. The various grades available under a given trademarks represent differences in molecular weight and viscosity.

In one aspect, the carrier is a mixture of polyethylene glycols having a molecular weight of from about 100 to about 20,000. In another aspect, the carrier is a mixture of polyethylene glycols having a molecular weight of from about 1000 to about 10,000. In some aspects, the polyethylene glycol is polyethylene glycol 1450, polyethylene glycol 3350 or polyethylene glycol 8000, or a mixture thereof.

It is to be understood that adding additional components to the polymers may be contemplated which is envisioned within the scope of the invention provided there is no deleterious effect on the overall composition and effective therapeutic effectiveness of the medication. Thus, in another aspect, the carrier may include a mixture of polyvinylpyrrolidones having a mean molecular weight ranging from 2,500 to 3,000,000 or more. There are many commercially available polyvinylpyrrolidone polymers suitable for the purposes of this invention. In another aspect, the carrier may be a cellulose ether. Exemplary cellulose ethers may include, without limitation, hydroxyalkyl cellulose (such as hydroxyethyl cellulose, hydroxypropyl cellulose, hydroxypropylmethyl cellulose, cellulose acetate trimellitate etc), and carboxyalkyl celluloses (such as carboxymethyl cellulose, carboxyethyl cellulose, etc) or a mixture thereof. In some aspects, the carrier may include adjuvants such as opacifiers, bulking agents, sweeteners, stabilizing agents, etc. Examples of opacifiers may include, without limitation, titanium dioxide, Talc, calcium carbonate, behenic acid, and cetyl alcohol. Examples of bulking agents may include, without limitation, starch, microcrystalline cellulose, calcium sulfate, calcium phosphate, and lactose. Non-limiting examples of sweeteners may include aspartame, saccharin, sodium cyclamate and Xylitol. Examples of stabilizing agents may include alginic acid glycerylmonostearate, hydroxypropyl cellulose, magnesium, aluminum silicate, and propylene glycol.

The solid oral dosage forms of the present invention can be processed into an immediate release or a sustained release dosage form. Immediate release dosage forms may release the atomoxetine agent in a fairly short time, for example, within a few minutes to within a few hours. Sustained release dosage forms may release the atomoxetine, agent over a period of several hours, for example, up to 24 hours or longer, if desired. In either case, the delivery can be controlled to be substantially at a certain predetermined rate over the period of delivery. The dissolution rate of the oral dosage form can be influenced by including adjuvants such as surfactants to the dosage form. Examples of suitable surfactants may include, without limitation, sodium lauryl sulfate, glyceryl monooleate, sorbitan ester, docusate sodium, and cetrimide. The surfactant may constitute from about 0.1% to about 5% by weight of the dosage form. In one aspect, for example, a polyethylene glycol dosage form of the present invention as described herein may comprise about 2.5% by weight sodium lauryl sulfate to provide an immediate release dosage form.

In the case of sustained release dosage forms, additional solid carriers can be used, including, but not limited to, gums, acrylic resins or a mixture thereof.

Additionally, the tablet or caplet dosage forms can be scored to facilitate easy break-off to adjust the dosage as needed. The tablets can also be multi-layered, each layer representing a different drug or a different concentration of the same drug. Alternatively, the dosage forms of the present invention can be prepared as unit dosage forms which are intended to deliver one therapeutically effective dose per administration. General methods and equipment for preparing tablets, capsules, pellets, and powders are well-known in the art. See, Remington, supra, Chapter 45, which is incorporated herein by reference.

In another aspect, the atomoxetine formulation may be in liquid oral dosage form. Liquid oral dosage forms may include, without limitation, emulsions, dispersions, suspensions, etc. Descriptions of liquid oral dosage forms can be found in Remington, supra, Chapter 39, which is incorporated herein by reference.

The amount of an atomoxetine agent to be orally administered may be measured according to several different parameters. In one aspect, the amount of the atomoxetine agent administered may be an amount sufficient to achieve a therapeutic effect. The amount required to obtain a therapeutic effect may vary depending on a number of factors, including the activity or potency of the specific atomoxetine agent selected, as well as physiological variations among subjects as to drug tolerance and general metabolic issues. In one aspect, behavioral variation can provide some measure of therapeutic effectiveness. As such, it is well within the knowledge of those skilled in the art and in view of the present disclosure to determine dosages of atomoxetine agents that are therapeutically effective for a given subject. In one aspect, at least about 1 mg of an atomoxetine agent can be administered to achieve therapeutic effectiveness. In another aspect, from about 1 mg to about 200 mg can be administered. In yet another aspect, from about 60 mg to about 200 mg can be administered. In a further aspect, from about 100 mg to about 175 mg can be administered. In yet a further aspect, from about 150 mg to about 200 mg can be administered. In another aspect, at least from about 1 mg to about 60 mgs can be administered. In yet another aspect, at least from about 2 mg to about 40 mgs can be administered. In yet another aspect, from about 2 mg to about 25 mgs can be administered. In a further aspect, from about 30 mg to about 50 mg can be administered. In yet a further aspect, up to at about 100 mg can be administered. It should be noted that dosages may be highly variable depending on the potency of the atomoxetine agent, and as such, the previously disclosed dosages are not to be limiting in any way.

In addition to oral formulations, the atomoxetine agent formulations of the present invention may be administered non-orally. In one aspect, such non-oral administration may include a transdermal formulation. Any pharmaceutically acceptable transdermal formulation and method for administering an atomoxetine agent that does not interfere with the drug's therapeutic effects may be used for achieving the desired aspects of the present invention. The transdermal drug delivery system of the present invention may take a variety of well-known delivery formulations, including but not limited to, transdermal patches such as adhesive matrix patches, liquid reservoir system (LRS) patches, transmucosal patches or tablets, and topical formulations, such as creams, lotions, ointments, gels, pastes, mousses, aerosols, sprays, waxes, balms, suppositories, etc.

When presented in the form of a transdermal patch, the transdermal drug delivery system of the present invention may include various structural components, as is known in the art. For example, in the case of an adhesive matrix patch, a distal backing is often laminated to a matrix polymer layer. Such a distal backing defines the side of the matrix patch that faces the environment, i.e., distal to the skin or mucosa. The backing layer functions to protect the matrix polymer layer and drug/enhancer composition and to provide an impenetrable layer that prevents loss of drug to the environment. Thus, the material chosen for the backing should be compatible with the polymer layer, drug, and other components such as an enhancer, and should be minimally permeable to any components of the matrix patch. In one aspect, the backing may be opaque to protect components of the matrix patch from degradation from exposure to ultraviolet light. In another aspect, the backing may be transparent in order to minimize the visibility of the patch when applied. Furthermore, the backing should be capable of binding to and supporting the polymer layer, yet should be pliable enough to accommodate the movements of a person using the matrix patch.

Suitable materials for the backing include, but are not limited to: metal foils, metalized polyfoils, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene and styrene-isoprene copolymers, polyethylene, and polypropylene. Additionally, the backing may include various foams, such as closed cell foams. Examples may include, without limitation, polyolefin foams, polyvinyl chloride foams, polyurethane foams, polyethylene foams, etc. In one aspect of the invention, the backing layer may have a thickness of about 0.0005 to 0.1 inch.

In one general aspect, the transdermal drug delivery system of the present invention can comprise a pharmaceutically acceptable carrier intended to contain the atomoxetine compound and any other components included in the formulation. A number of pharmaceutically acceptable carriers are known to those of ordinary skill in the art and may be used in connection with the present invention.

Further, a release liner may be temporarily provided upon the proximal side (side to adhere to the skin) of an adhesive layer. Such a liner provides many of the same functions as the backing layer, prior to adhesion of the patch to the skin. In use, the release liner is peeled from the adhesive layer just prior to application and discarded. The release liner can be made of the same materials as the backing layer, or other suitable films coated with an appropriate release surface.

Pharmaceutically acceptable carriers for use when the transdermal formulations of the present invention take the embodiment of an LRS patch may be any suitable viscous material known to those skilled in the art of transdermal drug delivery. Such carriers are typically a fluid of desired viscosity, such as a gel or ointment, which is formulated for confinement in a reservoir having an impermeable backing and a skin contacting permeable membrane, or membrane adhesive laminate providing diffusional contact between the reservoir contents and the skin. Such a viscous carrier may contain the atomoxetine compound to be transdermally delivered, as well as other optional components of the transdermal formulation.

Pharmaceutically acceptable carriers suitable for use when the present invention takes the embodiment of a transdermal matrix patch are also known to those of ordinary skill in the art. In one aspect, the present invention contemplates various structural types of transdermal matrix patches. For example, monolithic systems where the drug and enhancer are contained directly in a single pressure sensitive adhesive layer, as well as systems containing one or more polymeric reservoirs in addition to a pressure sensitive adhesive layer may be utilized. In aspects comprising systems having multiple layers/laminates, a rate controlling member may be included. Generally, a rate controlling member is located between a reservoir layer and the skin. In those aspects including a delivery layer and a reservoir layer, the rate controlling member may be adhered between a proximal side of the reservoir layer, and a distal side of the delivery layer. The rate controlling member is provided for the purpose of metering, or controlling, the rate at which drug and/or permeation enhancer migrates from the storage layer into the delivery layer. As noted herein, in one aspect of the present invention, various levels of permeation enhancement may be used to increase the delivery rate of the drug, and thus be used to vary other parameters, such as patch size, etc.

In one aspect, the pharmaceutically acceptable carrier used in a matrix patch can be a biocompatible polymer. Various general categories of biocompatible polymers are known, including, without limitation, rubbers; silicone polymers and copolymers; acrylic polymers and copolymers; and mixtures thereof. In one aspect, the biocompatible polymer can be a rubber, including natural and synthetic rubbers. One specific example of a useful rubber is a plasticized styrene-rubber block copolymer. In another aspect, the biocompatible polymer can include silicon polymers, polysiloxanes, and mixtures thereof. In yet another aspect, the biocompatible polymer can include acrylic polymers, polyacrylates, and mixtures thereof. In a further aspect, the biocompatible polymer can include vinyl acetates, ethylene-vinyl acetate copolymers, polyurethanes, plasticized polyether block amide copolymers, and mixtures thereof. In one specific aspect, the biocompatible polymer can include an acrylic copolymer adhesive such as copolymers of 2-ethylhexylacrylate and n-vinyl pyrrolidone adhesives.

In one aspect, the biocompatible polymer of the pharmaceutically acceptable carrier can be suitable for long-term (e.g., greater than 1 day, maybe about 3-4 days, or longer such as 7 days, or even 1-4 weeks) contact with the skin. In another aspect, the biocompatible polymer of the carrier is suitable for a short-term administration (e.g., for a few minutes to a few hours, less than or equal to 1 day). Such biocompatible polymers must be physically and chemically compatible with the atomoxetine agent, and with any carriers and/or vehicles or other additives incorporated into the formulation. In one aspect of the invention, the biocompatible polymers of the pharmaceutically acceptable carrier can include polymeric adhesives. Example of such adhesives can include without limitation, acrylic adhesives including cross-linked and uncross-linked acrylic copolymers; vinyl acetate adhesives; natural and synthetic rubbers including polyisobutylenes, neoprenes, polybutadienes, and polyisoprenes; ethylenevinylacetate copolymers; polysiloxanes; polyacrylates; polyurethanes; plasticized weight polyether block amide copolymers, and plasticized styrene-rubber block copolymers or mixtures thereof. In a further aspect of the invention, contact adhesives for use in the pharmaceuticaljy acceptable carrier layer are acrylic adhesives, such as DUROTAK® 87-2888 adhesive (National Starch & Chemical Co., Bridgewater, N.J.); and polyisobutylene adhesives such as ARCARE®. MA-24 (Adhesives Research, Glen Rock, Pa.) and ethylene vinyl acetate copolymer adhesives. In yet another aspect, gel-type or “hydrogel” adhesives are contemplated for use. See for example, U.S. Pat. No. 5,827,529 which is incorporated herein by reference. Those of ordinary skill in the art will appreciate that the specific type and amount of adhesive polymer used may be selected depending upon the desired specific characteristics of the final product. However, in one aspect, the amount of adhesive polymer in the adhesive matrix layer may be at least about 50% w/w of the adhesive layer. In another aspect, the amount may be at least about 60% w/w of the adhesive layer. In yet another aspect, the amount may be at least about 85% w/w of the adhesive layer. In a further aspect, the amount may be at least about 90% w/w of the adhesive layer. In an additional aspect, the amount may be from about 50% w/w to about 95% w/w of the adhesive layer.

Transdermal matrix patches may be utilized in various sizes, depending on the atomoxetine dosage in the patch and the desired rate of delivery. In one aspect, transdermal patches may be from about 0.5 cm² to about 200 cm² in size. In another aspect, transdermal patches may be from about 5 cm² to about 75 cm² in size. In yet another aspect, transdermal patches may be from about 10 cm² to about 100 cm² in size. In a further aspect, transdermal patches may be from about 50 cm² to about 100 cm² in size. In yet a further aspect, transdermal patches may be from about 0.5 cm² to about 100 cm² in size. In an additional aspect, transdermal patches may be from about 100 cm² to about 200 cm² in size. In yet an additional aspect, transdermal patches may be from about 10 cm² to about 50 cm² in size.

Various pharmaceutically acceptable carriers which are known to those of ordinary skill in the art may be used when the transdermal formulations of the present invention take the embodiment of a topical formulation. In one aspect, the topical carrier can be an ointment including an atomoxetine agent. An ointment is a semisolid pharmaceutical preparation based on well known materials such as oleaginous bases, lanolins, emulsions, or water-soluble bases. Preparation of ointments is well known in the art such as described in Remington, supra, Chapter 44, which is incorporated herein by reference. Such preparations often contain petrolatum or zinc oxide together with a drug. Oleaginous ointment bases suitable for use in the present invention include generally, but are not limited to, vegetable oils, animal fats, and semisolid hydrocarbons obtained from petroleum. Absorbent ointment bases of the present invention may contain little or no water and may include components such as, but not limited to, hydroxystearin sulfate, anhydrous lanolin and hydrophilic petrolatum. Emulsion ointment bases of the present invention are either water-in-oil (W/O) emulsions or oil-in-water (O/W) emulsions, and may include, but are not limited to, cetyl alcohol, glyceryl monostearate, lanolin, polyalkylsiloxanes, and stearic acid. Water-soluble ointment bases suitable for use in the present invention may be prepared from polyethylene glycols of varying molecular weight.

In another aspect of the present invention, the topical carrier can be a cream including an atomoxetine agent. Creams are a type of ointment which are viscous liquids or semisolid emulsions, either oil-in-water or water-in-oil, as is well known in the art. Cream bases may be soluble in water, and contain an oil phase, an emulsifier, an aqueous phase, and the active agent. In a detailed aspect of the present invention, the oil phase may be comprised of petrolatum and a fatty alcohol such as cetyl or stearyl alcohol. In another detailed aspect of the present invention, the aqueous phase may exceed the oil phase in volume, and may contain a humectant. In another detailed aspect of the present invention, the emulsifier in a cream formulation may be a nonionic, anionic, cationic or amphoteric surfactant.

In another aspect of the present invention, the topical carrier can be a lotion including an atomoxetine agent. A lotion is an ointment which may be a liquid or semi-liquid preparation in which solid particles, including the active agent, are present in a water or alcohol base. Lotions suitable for use in the present invention may be a suspension of solids or may be an oil-in-water emulsion. In another aspect of the present invention, lotions may also contain suspending agents which improve dispersions or other compounds which improve contact of the active agent with the skin, e.g., methylcellulose, sodium carboxymethylcellulose, or similar compounds.

In yet another aspect of the present invention, a topical carrier can be a paste including an atomoxetine agent. Pastes of the present invention are ointments in which there are significant amounts of solids which form a semisolid formulation in which the active agent is suspended in a suitable base. In a detailed aspect of the present invention, pastes may be formed of bases to produce fatty pastes or made from a single-phase aqueous gel. Fatty pastes suitable for use in the present invention may be formed of a base such as petrolatum, hydrophilic petrolatum or the like. Pastes made from single-phase aqueous gels suitable for use in the present invention may incorporate cellulose based polymers such as carboxymethylcellulose or the like as a base.

In another aspect of the present invention, a topical gel may be prepared that includes an atomoxetine agent. A gel prepared in accordance with the present invention may be a preparation of a colloid in which a disperse phase has combined with a continuous phase to produce a viscous product. The gelling agent may form submicroscopic crystalline particle groups that retain the solvent in the interstices. As will be appreciated by those working in art, gels are semisolid, suspension-type systems. Single-phase gels can contain organic macromolecules distributed substantially uniformly throughout a carrier liquid, which may be aqueous or non-aqueous and may contain an alcohol or oil.

The exact amount of an atomoxetine agent to be included in a transdermal formulation to achieve a therapeutically effective amount may also be highly variable, depending on the potency of the atomoxetine agent, the specific type of transdermal formulation being employed, as well as physiological variations among subjects as to drug tolerance and general metabolic issues. It is also noted, however, that the dosage required to provide a therapeutically effective amount can be readily determined by one of ordinary skill in the art. Further, considerations for drug load may also be made in view of specifically desired properties for the transdermal formulation, such as size, delivery rate, and duration of administration, and may range from subsaturated to supersaturated concentrations. However, in one aspect, the amount of an atomoxetine agent may be from about 0.1% w/w to about 50% w/w of the formulation. In a further aspect, the amount of an atomoxetine agent may be from about 1% w/w to about 20% w/w of the formulation. In another aspect, the amount of an atomoxetine agent may be from about 2% w/w to about 10% w/w. In an additional aspect, an atomoxetine agent amount may be about 5% w/w of the formulation. As has been previously noted, dosages may be highly variable depending on the potency of the atomoxetine agent, and as such, the previously disclosed dosages are not to be limiting in any way.

The administration dosage of the atomoxetine agent may also be characterized in terms of blood serum levels. In one aspect, for example, an atomoxetine agent may be transdermally administered in an amount sufficient to achieve and sustain a therapeutically effective blood serum level for at least about one day. In another aspect, an atomoxetine agent may be transdermally administered in an amount sufficient to achieve and sustain a therapeutically effective blood serum level for less than about one day. In yet another aspect, an atomoxetine agent may be transdermally administered in an amount sufficient to achieve and sustain a therapeutically effective blood serum level for from about one day to about 7 days. In a further aspect, an atomoxetine agent may be transdermally administered in an amount sufficient to achieve and sustain a therapeutically effective blood serum level for from about 7 days to about 14 days. In a yet a further aspect, an atomoxetine agent may be transdermally administered in an amount sufficient to achieve and sustain a therapeutically effective blood serum level for from about 1 day to about 14 days.

The transdermal formulations of the present invention can also be formulated as sustained release formulations that administer therapeutically effective amounts of an atomoxetine agent over an extended period of time. As such, in one aspect, the sustained delivery period of the agent may be for at least 7 days. In another aspect, the sustained delivery period may be at least 5 days. In a further aspect, the sustained delivery period may be at least 3 days. In another aspect, the sustained delivery period may be at least one day. In yet another aspect, the sustained delivery period may be less than one day. In a further aspect, the sustained delivery period may be from about 1 to about 4 weeks.

In addition to containing an atomoxetine agent, the pharmaceutically acceptable carriers of the transdermal formulations recited herein may include a number of other additives, such as diluents, permeation enhancers, excipients, emollients, plasticizers, skin irritation reducing agents, stabilizing compounds, or a mixture thereof. These types of components, as well as others not specifically recited, are well known in the art for inclusion in various transdermal formulations, and may be added as desired to the transdermal drug delivery system of the present invention in specific types and amounts in order to achieve a desired result.

Furthermore, when the atomoxetine agent to be delivered is susceptible to acid catalyzed degradation, carriers that contain no acid functional groups, and that do not form any acid functional groups upon storage can be used in order to improve the stability of the formulation. One specific example of such a carrier is an ethylhexylacrylate polymer, as described in U.S. Pat. No. 5,780,050, which is incorporated by reference herein.

In addition to the atomoxetine agent, the transdermal formulations of the present invention may also include a permeation enhancer, or mixture of permeation enhancers in order to increase the permeability of the skin to the atomoxetine agent. For example, useful permeation enhancers may include, without limitation, fatty acids, fatty acid esters, fatty alcohols, fatty acid esters of lactic acid or glycolic acid, glycerol tri-, di-, and monoesters, triacetin, short chain alcohols, and mixtures thereof. In one specific aspect, the permeation enhancer may include lauryl alcohol, isopropyl myristate, or a combination of lauryl alcohol and isopropyl myristate. In other aspects, specific species or combinations of species may be selected from the above listed classes of compounds by one skilled in the art, in order to optimize enhancement of the particular atomoxetine agent employed.

The formulations of the present invention may also include metabolic inhibitors to increase the potency of the administered atomoxetine agent. Because various atomoxetine agents appear to be primarily metabolized by various cytochrome P450 enzymes, selective inhibition of certain enzymes may thus increase the potency of the administered atomoxetine agent by reducing metabolic activity. As such, in one aspect, a P450-mediated reaction inhibitor may be administered to a subject. The P450-mediated reaction can be any enzymatic pathway responsible for metabolism on an atomoxetine agent. Furthermore, the particular P450-mediated reaction may be selected based on the particular atomoxetine agent administered. Thus the inhibitor can be any inhibitor known to reduce the activity of the particular P450-mediated reaction. For example, and without limitation, CYP2A6 may be inhibited by coumarin, CYP2C9 by sulfaphenazole, CYP2C19 by S-mephenyloin, CYP2D6 by quinidine, CYP3A by ketoconazole, etc. In one aspect, quinidine may be useful as a P450-mediated reaction inhibitor due to the enzymatic activity of CYP2D6 in metabolizing various atomoxetine agents.

Various temporal orders of administering the atomoxetine agent and the inhibitor are possible, and any such order of administration that obtains a therapeutically result is considered to be within the scope of the present invention. In one aspect, the atomoxetine agent and the inhibitor can be administered concomitantly, either as a single composition or as separate compounds. Such concurrent administration is intended to include application of each of the compounds at essentially the same time. In those aspects where the inhibitor is administered separately from the atomoxetine agent, the inhibitor can be administered prior to, following, or both prior to and following the administration of the atomoxetine agent.

### EXAMPLES

The following examples of formulations of atomoxetine agents are provided to promote a more clear understanding of certain embodiments of the present invention, and are in no way meant as a limitation thereon.

### Example 1

### Preparation of N-Methylatomoxetine N-Oxide Adhesive Matrix Patch

A general method of preparing transdermal adhesive matrix patches is described by U.S. Pat. Nos. 5,227,169, and 5,212,199, which are incorporated by reference in their entirety. Following this general method, the N-methylatomoxetine N-oxide patches of this invention are prepared as follows: N-methylatomoxetine N-oxide, triacetin (Eastman Chemical Co., Kingsport, N.Y.) and 87-2888 acrylic copolymer adhesives (National Starch and Chemical Co., Bridgewater, N.J.) are mixed into a homogenous solution and coated at 6 mg/cm² (dried weight) onto a silicone treated polyester release liner (Rexham Release, Chicago, Ill.) using a two zone coating/drying/laminating oven (Kraemer Koating, Lakewood, N.J.) to provide a final N-methylatomoxetine N-oxide adhesive matrix containing 15.4%, 9.0%, and 75.6% by weight N-methylatomoxetine N-oxide, triacetin and acrylic copolymer adhesive, respectively. A fifty micron thick polyethylene backing film (3M, St. Paul, Minn.) is subsequently laminated onto the dried adhesive surface of the N-methylatomoxetine N-oxide containing adhesive matrix and the final laminate structure is die cut to provide patches ranging in size from 13 cm² to 39 cm² patches.

### Example 2

### Preparation of Topical N-Methylatomoxetine N-Oxide Formulation

Topically applied N-methylatomoxetine N-oxide containing gel may be used to deliver N-methylatomoxetine N-oxide in accordance with the method of the present invention. A general method of preparing a topical gel is known in the art. Following this general method, a topical gel comprising N-methylatomoxetine N-oxide is prepared as follows:

95% ethanol (USP) is diluted with water (USP), glycerin (USP), and glycerol monooleate (Eastman Chemical, Kingsport N.Y.) to provide a final solution at ethanol/water/glycerin/glycerol monooleate percent ratios of 35/59/5/1, respectively. N-methylatomoxetine N-oxide is then dissolved into the above solution to a concentration of 10 mg/gram. The resultant solution is then gelled with 1% hydroxypropyl cellulose (Aqualon, Wilmington, Del.) to provide a final N-methylatomoxetine N-oxide gel. One to two grams of the above gel is applied topically to approximately 200 cm² surface area on the chest, torso, and or arms to provide topical administration of N-methylatomoxetine N-oxide.

### Example 3

### Preparation of an Oral N-Methylatomoxetine N-Oxide Formulation

A composition for preparing a 10 mg N-methylatomoxetine N-oxide oral formulation is provided in Table 1.

TABLE 1
10 mg N-methylatomoxetine N-oxide Formulation

| Component | mg/unit | % (by weight) |
|---|---|---|
| N-methylatomoxetine N-oxide | 10.0 | 5.00 |
| Polyethylene glycol 1000, NF | 15.00 | 7.5 |
| Polyethylene glycol 1450, NF | 31.00 | 15.50 |
| Polyethylene glycol 3350, NF | 76.00 | 38.00 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

### Example 4

### Preparation of an Oral 4-hydroxyatomoxetine Formulation

A composition for preparing a 10 mg 4-hydroxyatomoxetine oral formulation is provided in Table 2.

TABLE 2
10 mg 4-hydroxyatomoxetine Formulation

| Component | mg/unit | % (by weight) |
|---|---|---|
| 4-hydroxyatomoxetine | 10.0 | 5.00 |
| Polyethylene glycol 400, NF | 5.00 | 2.5 |
| Polyethylene glycol 1000, NF | 15.00 | 7.5 |
| Polyethylene glycol 1450, NF | 31.00 | 15.50 |
| Polyethylene glycol 3350, NF | 71.00 | 35.50 |
| Polyethylene glycol 8000, NF | 16.00 | 8.00 |
| Pregelatinized starch, NF | 49.00 | 24.50 |
| Titanium dioxide, NF | 1.00 | 0.50 |
| Zinc stearate, NF | 2.00 | 1.00 |
| TOTAL | 200.0 | 100.0% |

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of treating or preventing a condition in a subject for which an atomoxetine compound is effective, comprising:
administering a therapeutically effective amount of an atomoxetine agent to the subject.

2. The method of claim 1, wherein the atomoxetine agent is a compound of Formula I: 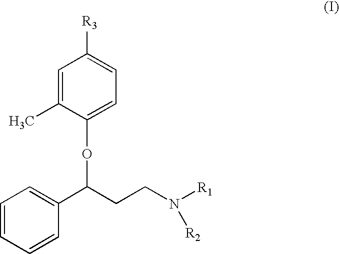
or an isomer, stereoisomer, enantiomer, tautomer, analog, salt, or combination thereof, wherein:
R₁ and R₂ are independently a branched or unbranched C₁-C₄ alkyl or a branched or unbranched C₁-C₄ N-oxide, including respective tertiary oxides;
R₃ is —H or —OR₄; and
R₄ is alkyl or branch chain alkyl of C1-C18, a substituted or unsubstituted phenyl ring, —C₆H₉O₆, or —H.

3. The method of claim 2, wherein R₁ and R₂ are the same.

4. The method of claim 2, wherein R₁ and R₂ are different.

5. The method of claim 1, wherein the atomoxetine agent is an atomoxetine metabolite.

6. The method of claim 1, wherein the atomoxetine agent is a compound of Formula II: 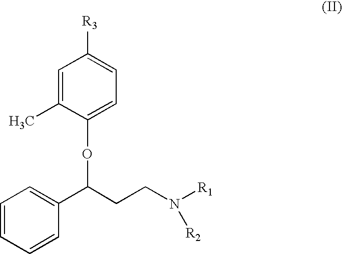
or an isomer, stereoisomer, enantiomer, tautomer, analog, salt, or combination thereof, wherein:
R₁ is —H or a CH₃;
R₂ is —CH₃ or —H;
R₃ is —H or —OR₄ when R₂ is —H and R₃ is OR₄ when R₂ is —CH₃; and
R₄ is alkyl or branch chain alkyl of C₁-C₁₈, a substituted or unsubstituted phenyl ring, ˜C₆H₉O₆, or —H.

7. The method of claim 1, wherein the atomoxetine agent is an atomoxetine prodrug.

8. The method of claim 1, wherein the condition is selected from the group consisting of attention-deficit/hyperactivity disorders, asthma, allergic rhinitis, cognitive failure, tic disorders, depression, resistant depression with psychotic features, motor deficit after stroke, memory disorders, obesity, Tourette's syndrome, traumatic brain injury, bipolar disorder, anxiety, narcolepsy, nocturnal enuresis, fibromyalgia syndrome, schizophrenia, post traumatic stress disorder, and combinations and related disorders thereof.

9. The method of claim 8, wherein the condition is attention deficit/hyperactivity disorder, a cognitive decline, a depressive disorder, or a post traumatic stress disorders.

10. The method of claim 1, wherein the atomoxetine agent is an atomoxetine prodrug.

11. The method of claim 1, wherein the atomoxetine agent is an atomoxetine metabolite.

12. The method of claim 1, wherein administering the atomoxetine agent further includes administering the atomoxetine agent orally.

13. The method of claim 1, wherein administering the atomoxetine agent further includes administering the atomoxetine agent non-orally.

14. The method of claim 13, wherein administering the atomoxetine agent non-orally further includes administering the atomoxetine agent parenterally.

15. The method of claim 13, wherein administering the atomoxetine agent non-orally further includes administering the atomoxetine agent transdermally.

16. The method of claim 1, further including administering a P450-mediated reaction inhibitor to the subject.

17. The method of claim 16, wherein the P450-mediated reaction inhibitor is a CYP2D6 inhibitor.

18. The method of claim 17, wherein the CYP2D6 inhibitor is quinidine.

19. The method of claim 16, wherein the P450-mediated reaction inhibitor is administered prior to, concurrently with, or following the atomoxetine agent.

20. The method of claim 16, wherein the P450-mediated reaction inhibitor is administered concurrently with the atomoxetine agent.

21. The method of claim 20, wherein the P450-mediated reaction inhibitor and the atomoxetine agent are administered as a single composition.

22. The method of claim 16, wherein the P450-mediated reaction inhibitor is administered prior to and following the atomoxetine agent.

23. An atomoxetine agent formulation for treating or preventing a condition, comprising:
a therapeutically affective amount of an atomoxetine agent in combination with a pharmaceutically acceptable carrier.

24. The formulation of claim 23, wherein the atomoxetine agent is an atomoxetine metabolite.

25. The formulation of claim 23, wherein the atomoxetine agent is an atomoxetine prodrug.

26. The formulation of claim 23, wherein the atomoxetine agent is a compound of Formula III: 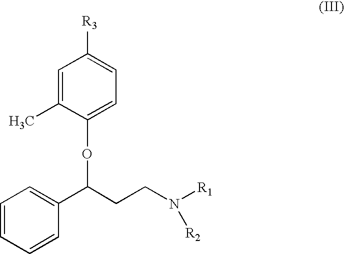
or an isomer, stereoisomer, enantiomer, tautomer, analog, salt, or combination thereof, wherein:
R₁ and R₂ are independently a branched or unbranched C₁-C₃ alkyl or a branched or unbranched C₁-C₃ N-oxide, including respective tertiary oxides;
R₃ is —H or —OR₄; and
R₄ is alkyl or branch chain alkyl of C₁-C₁₈, a substituted or unsubstituted phenyl ring, —C₆H₉O₆, or —H.

27. The formulation of claim 26, wherein R₁ and R₂ are the same.

28. The formulation of claim 26, wherein R₁ and R₂ are different.

29. The formulation of claim 23, wherein the atomoxetine agent is a compound of Formula IV: 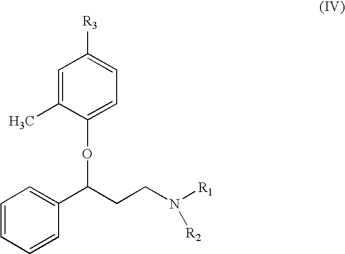
or an isomer, stereoisomer, enantiomer, tautomer, analog, salt, or combination thereof, wherein:
R₁ is —H;
R₂ is —CH₃ or —H;
R₃ is —H or —OR₄ when R₂ is —H and R₃ is —OR₄ when R₂ is —CH₃; and
R₄ is alkyl or branch chain alkyl of C₁-C₁₈, a substituted or unsubstituted phenyl ring, —C₆H₉O₆, or —H.

30. The formulation of claim 23, Wherein the atomoxetine agent is a compound of Formula V:
(V) 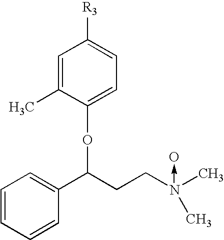
or an isomer, stereoisomer, enantiomer, tautomer, analog, salt, or combination thereof, wherein:
R₃ is —H or —OR₄; and
R₄ is alkyl or branch chain alkyl of C1-C18, a substituted or unsubstituted phenyl ring, —C₆H₉O₆, or —H.

31. The formulation of claim 23, further comprising a P450-mediated reaction inhibitor.

32. The formulation of claim 31, wherein the P450-mediated reaction inhibitor is a CYP2D6 inhibitor.

33. The formulation of claim 32, wherein the CYP2D6 inhibitor is quinidine.

34. The formulation of claim 31, wherein the P450-mediated reaction inhibitor and the atomoxetine agent are a composition.

35. The formulation of claim 23, wherein the pharmaceutically acceptable carrier is a pharmaceutically acceptable non-oral carrier.

36. The formulation of claim 35, wherein the pharmaceutically acceptable non-oral carrier is a pharmaceutically acceptable transdermal carrier.

37. The formulation of claim 36, wherein the pharmaceutically acceptable transdermal carrier is a biocompatible polymer.

38. The formulation of claim 37, wherein the biocompatible polymer is a member selected from the group consisting of: rubbers; silicone polymers and copolymers; acrylic polymers and copolymers; and mixtures thereof.

39. The formulation of claim 37, wherein the biocompatible polymer is a rubber selected from the group consisting of: natural and synthetic rubbers, plasticized styrene-rubber block copolymers, and mixtures thereof.

40. The formulation of claim 37, wherein the biocompatible polymer is a member selected from the group consisting of: silicone polymers, polysiloxanes, and mixtures thereof.

41. The formulation of claim 37, wherein the biocompatible polymer is a member selected from the group consisting of: acrylic polymers, polyacrylates, and mixtures thereof.

42. The formulation of claim 37, wherein the biocompatible polymer is a member selected from the group consisting of vinyl acetates, ethylene-vinyl acetate copolymers, polyurethanes, plasticized polyether block amide copolymers, and mixtures thereof.

43. The formulation of claim 36, wherein the pharmaceutically acceptable transdermal carrier comprises a viscous material suitable for use as a liquid reservoir.

44. The formulation of claim 43, wherein the viscous material forms a gel.

45. The formulation of claim 36, further comprising an ingredient selected from the group consisting of: diluents, permeation enhancers, excipients, emollients, plasticizers, skin irritation reducing agents, stabilizing compounds, and mixtures thereof.

46. The formulation of claim 36, wherein the formulation is a transdermal patch.

47. The formulation of claim 46, wherein the transdermal patch is a transdermal matrix patch.

48. The formulation of claim 46, wherein the transdermal patch is a liquid reservoir patch.

49. The transdermal atomoxetine formulation of claim 36, wherein the formulation is a topical formulation.

50. The formulation of claim 49, wherein the topical formulation is in a form selected from the group consisting of creams, lotions, ointments, gels, pastes, mousses, aerosols, sprays, waxes, balms, suppositories, and mixtures or combinations thereof.

51. The formulation of claim 35, wherein the atomoxetine agent is from about 0.1% w/w to about 50% w/w of the non-oral formulation.

52. The formulation of claim 51, wherein the atomoxetine agent is from about 1% w/w to about 20% w/w of the non-oral formulation.

53. The formulation of claim 52, wherein the atomoxetine agent is from about 3% w/w to about 10% w/w of the non-oral formulation.

54. The formulation of claim 35, wherein the pharmaceutically acceptable non-oral carrier is a pharmaceutically acceptable parenteral carrier.

55. The formulation of claim 23, wherein the pharmaceutically acceptable carrier is a pharmaceutically acceptable oral carrier.

56. The formulation of claim 55, wherein the pharmaceutically acceptable oral carrier is a solid carrier.

57. The formulation of claim 55, wherein the pharmaceutically acceptable oral carrier is a liquid carrier.

58. The formulation of claim 55, further comprising an ingredient selected from the group consisting of: diluents, binders, lubricants, disintegrants, coloring agents, flavoring agents, enhancers, excipients, plasticizers, stabilizing compounds, and mixtures thereof.

59. The formulation of claim 55, wherein the atomoxetine agent is from about 0.1% w/w to about 50% w/w of the oral formulation.

60. The formulation of claim 55, wherein the atomoxetine agent is from about 1% w/w to about 20% w/w of the oral formulation.

61. The formulation of claim 55, wherein the atomoxetine agent is from about 3% w/w to about 10% w/w of the oral formulation.

62. A transdermal atomoxetine agent formulation, comprising:
a pressure sensitive acrylic polymer in an amount of about 70% w/w of the transdermal formulation;
N-ethylatomoxetine in an amount of about 5% w/w of the transdermal formulation;
polyvinylpyrrolidone in an amount of about 10% w/w of the transdermal formulation;
a penetration enhancer in an amount of about 20% w/w of the transdermal formulation selected from the group consisting of lower chain (C2 to C4) alcohols, lower chain diols such as propylene glycol- and di-propylene glycol, triacetin, glycerol monooleate, glycerol monolaurate, oleic alcohol, lauryl alcohol, isopropyl myristate, sorbitan esters, and combinations thereof; and
quinidine in an amount of about 0.1% w/w or greater of the transdermal formulation.

63. An oral atomoxetine agent formulation, comprising:
polyethylene glycol in an amount of about, from about 20% w/w to about 25% w/w of the oral formulation;
N-ethylatomoxetine in an amount of about 5% w/w of the oral formulation; and
quinidine in an amount of about 0.1% w/w or greater of the oral formulation.
